SEQUENCE LISTING (15 sequences)

SEQ ID NO: 1 - DNA, 25 bp, synthetic construct
  source 1..25: mol_type = other DNA
tggtagttggagcttgtggcgtagg

SEQ ID NO: 2 - DNA, 21 bp, synthetic construct
  source 1..21: mol_type = other DNA
ttttttttttccdnactacca

SEQ ID NO: 3 - DNA, 22 bp, synthetic construct
  source 1..22: mol_type = other DNA
ttttttttttcgccacdnagct

SEQ ID NO: 4 - DNA, 18 bp, synthetic construct
  source 1..18: mol_type = other DNA
ttttttttttacdnagct

SEQ ID NO: 5 - DNA, 15 bp, synthetic construct
  source 1..15: mol_type = other DNA
ttttttttttccdna

SEQ ID NO: 6 - DNA, 32 bp, synthetic construct
  source 1..32: mol_type = other DNA
agcttgtgcgtaggcdnagagtggccttcacg

SEQ ID NO: 7 - DNA, 20 bp, synthetic construct
  source 1..20: mol_type = other DNA
ttttttttttccacdnagct

SEQ ID NO: 8 - DNA, 19 bp, synthetic construct
  source 1..19: mol_type = other DNA
ttttttttttcacdnagct

SEQ ID NO: 9 - DNA, 20 bp, synthetic construct
  source 1..20: mol_type = other DNA
ccacdnagcttttttttttt

SEQ ID NO: 10 - DNA, 19 bp, synthetic construct
  source 1..19: mol_type = other DNA
cacdnagcttttttttttt

SEQ ID NO: 11 - DNA, 20 bp, synthetic construct
  source 1..20: mol_type = other DNA
ttttttttttcgtgdnaggc

SEQ ID NO: 12 - DNA, 19 bp, synthetic construct
  source 1..19: mol_type = other DNA
ttttttttttcgtgdnagg

SEQ ID NO: 13 - DNA, 31 bp, synthetic construct
  source 1..31: mol_type = other DNA
tcccdnacccactdnatggtgttggttacca

SEQ ID NO: 14 - DNA, 30 bp, synthetic construct
  source 1..30: mol_type = other DNA
tcccdnacccacttatggtgttggttacca

SEQ ID NO: 15 - DNA, 29 bp, synthetic construct
  source 1..29: mol_type = other DNA
tcccgacccacttatggtgttggtcacca

(19) United States
(12) Patent Application Publication
(10) Pub. No.: US 2025/0277258 A1
(43) Pub. Date: Sep. 4, 2025

(54) Heteromultivalent DNA-Functionalized Materials to Detect Genetic Mutations and Use in Diagnostic Applications

(71) Applicant: Emory University, Atlanta, GA (US)

(72) Inventors: Brendan Deal, Atlanta, GA (US); Khalid Salaita, Atlanta, GA (US); Rong Ma, Atlanta, GA (US); James Kindt, Atlanta, GA (US)

(21) Appl. No.: 18/858,695

(22) PCT Filed: Apr. 21, 2023
(86) PCT No.: PCT/US2023/066072

§ 371 Date: Oct. 21, 2024

Related U.S. Application Data
(60) Provisional application No. 63/333,147, filed on Apr. 21, 2022.

(51) Int. Cl. C12Q 1/6834; C12Q 1/6827; C12Q 1/70

(52) U.S. Cl. CPC C12Q 1/6834; C12Q 1/6827; C12Q 1/70; C12Q 2600/156

(57) ABSTRACT

This disclosure relates to heteromultivalent nucleic acid-functionalized surfaces, such as particles, and uses in optimizing hybridization specificity for targets containing one, two, or more mutations. In certain embodiments, heteromultivalent hybridization enables fine-tuned specificity for a single SNP and dramatic enhancements in specificity for two non-proximal SNPs empowered by cooperative binding. In certain embodiments, use of specified oligo lengths, spacer lengths, and binding orientation are contemplated. In certain embodiments, this disclosure provides for methods of discrimination between heterozygous cis and trans mutations and between different strains of a virus, e.g., the SARS-CoV-2 virus.

### CROSS-REFERENCE TO RELATED APPLICATIONS

This application claims the benefit of U.S. Provisional Application No. 63/333,147 filed Apr. 21, 2022. The entirety of this application is hereby incorporated by reference for all purposes.

### STATEMENT REGARDING FEDERALLY SPONSORED RESEARCH OR DEVELOPMENT

This invention was made with government support under CHE2004126 awarded by the National Science Foundation. The government has certain rights in the invention.

### INCORPORATION-BY-REFERENCE OF MATERIAL SUBMITTED AS AN XML FILE VIA THE OFFICE ELECTRONIC FILING SYSTEM

The Sequence Listing associated with this application is provided in XML format and is hereby incorporated by reference into the specification. The name of the XML file containing the Sequence Listing is 22114PCT.xml. The XML file is 14 KB, was created on Apr. 21, 2023, and is being submitted electronically via the USPTO patent electronic filing system.

### BACKGROUND

Detecting genetic mutations such as single nucleotide polymorphisms (SNPs) is helpful in therapeutically managing cancer therapies, perform genetic analyses, and distinguish similar viral strains. Traditionally, SNP sensing uses short oligonucleotide probes that differentially bind the SNP and wildtype targets. However, DNA hybridization-based techniques require precisely tuning the binding affinity of the probe to manage the inherent trade-off between specificity and sensitivity. High binding affinity results in improved sensitivity, allowing the detection of lower concentration oligonucleotides, but also leads to enhanced off-target binding and decreased discrimination between similar targets. Conversely, lowering target affinity can enhance specificity but lowers the limit of detection of an assay. Thus, there is an affinity “sweet spot” that maximizes the ratio between on- and off-target binding. Unfortunately, this optimized affinity is difficult to achieve, often resulting in poor discrimination for targets containing mismatches, such as single nucleotide polymorphisms (SNPs), which are biomedically relevant and challenging to identify. Tuning the affinity to maximize specificity can be achieved by changing the probe length. However, the problem with this strategy is that adding or removing a single base pair drastically changes affinity, resulting in low-precision affinity tuning. Adjusting temperature and ionic strength can precisely optimize probe affinity for SNP targets, but this approach fails when detecting multiple SNPs simultaneously in a multiplexed or microarray-type assay. Thus, there is a need to identify improvements.

Deal et al. report engineering DNA-functionalized nanostructures to bind nucleic acid targets heteromultivalently with enhanced avidity. J Am Chem Soc, 2020, 142(21): 9653-9660. See also WO2021/183485.

Fong et al. report the role of structural enthalpy in spherical nucleic acid hybridization. J Am Chem Soc, 2018, 140, 6226-6230.

Edwardson et al. report the transfer of molecular recognition information from DNA nanostructures to gold nanoparticles. Nature Chemistry, 2016, 8:162-170.

Estirado et al. report multivalent ultrasensitive interfacing of supramolecular 1D nanoplatforms. J. Am. Chem. Soc. 2019, 141, 18030-18037.

References cited herein are not an admission of prior art.

### SUMMARY

This disclosure relates to heteromultivalent nucleic acid-functionalized surfaces, such as particles, and uses in optimizing hybridization specificity for targets containing one, two, or more mutations. In certain embodiment, heteromultivalent hybridization enables fine-tuned specificity for a single SNP and dramatic enhancements in specificity for two non-proximal SNPs empowered by cooperative binding. In certain embodiments, use of specified oligo lengths, spacer lengths, and binding orientation are contemplated. In certain embodiments, this disclosure provides for methods of discrimination between heterozygous cis and trans mutations and between different strains of a virus, e.g., the SARS-CoV-2 virus.

In certain embodiments, this disclosure relates to particles or surfaces comprising: a single nucleic polymorph binding nucleic acid, wherein the single nucleic polymorph binding nucleic acid comprises a single nucleotide polymorph binding segment and a particle or surface binding segment attached to the particle or surface; wherein the particle or surface further comprises a tuning nucleic acid, wherein the tuning nucleic acid comprises a target binding segment and a particle or surface binding segment attached to the particle or surface.

In certain embodiments, the particle or surface further comprises a target nucleic acid, wherein the target nucleic acid comprises a single nucleotide polymorph segment having a single nucleotide polymorph, a target segment, and a spacer segment; wherein the target nucleic acid is bound to the particle or surface as the single nucleotide polymorph binding segment of the single nucleic polymorph binding nucleic acid is hybridized to the single nucleotide polymorph segment of the target nucleic acid; wherein the single nucleotide polymorph binding segment has a nucleobase that base pairs with the single nucleotide polymorph; wherein the target nucleic acid is bound to the particle or surface as the target binding segment of the tuning nucleic acid is hybridized to the target segment of the target nucleic acid.

In certain embodiments, this disclosure relates to methods of detecting the presence of a single nucleotide polymorph mutation comprising, contacting particles or surfaces reported herein with a sample from a subject comprising a target nucleic acid, wherein the target nucleic acid comprises a single nucleotide polymorph segment having a single nucleotide polymorph, a target segment, and a spacer segment; wherein the target nucleic acid binds the particle or surface as the single nucleotide polymorph binding segment of the single nucleic polymorph binding nucleic acid is hybridized to the single nucleotide polymorph segment of the target nucleic acid; wherein the single nucleotide polymorph binding segment has a nucleobase that base pairs with the single nucleotide polymorph; wherein the target nucleic acid is bound to the particle or surface as the target binding segment of the tuning nucleic acid is hybridized to the target segment of the target nucleic acid; and detecting that the target nucleic acid is bound to the particle or surface providing the presence of a single nucleotide polymorph mutation in the sample.

In certain embodiments, detecting that the target nucleic acid is bound to the particle or surface providing the presence of a single nucleotide polymorph mutation in the sample can be accomplished by one or a combination of the following steps; purifying the particles or surfaces; contacting the particles or surfaces with a probe that hybridizes with a single stranded segment of the target nucleic acid, e.g., in the spacer segment or segments flanking the single nucleotide polymorph binding segment or segment flanking target binding segments of the tuning nucleic acid and detecting the probe; denaturing the target nucleic acid from the particle or surface and amplifying target nucleic acid by PCR, and sequencing the target nucleic acid.

In certain embodiments, this disclosure contemplates particles or surfaces comprising: a first single nucleic polymorph binding nucleic acid, wherein the first single nucleic polymorph binding nucleic acid comprises a first single nucleotide polymorph binding segment and a particle or surface binding segment attached to the particle or surface; wherein the particle or surface further comprises a second single nucleic polymorph binding nucleic acid, wherein the second single nucleic polymorph binding nucleic acid comprises a second single nucleotide polymorph binding segment and a particle or surface binding segment attached to the particle or surface.

In certain embodiments, the particle or surface further comprises a target nucleic acid, wherein the target nucleic acid comprises a first single nucleotide polymorph segment, a second single nucleotide polymorph segment and a spacer segment; wherein the target nucleic acid is bound to the particle or surface as the first single nucleotide polymorph binding segment of the first single nucleic polymorph binding nucleic acid is hybridized to the first single nucleotide polymorph segment of the target nucleic acid; wherein the first single nucleotide polymorph binding segment has a nucleobase that base pairs with a first single nucleotide polymorph; wherein the target nucleic acid is bound to the particle or surface as the second single nucleotide polymorph binding segment of the second single nucleic polymorph binding nucleic acid is hybridized to the second single nucleotide polymorph segment of the target nucleic acid; wherein the second single nucleotide polymorph binding segment has a nucleobase that base pairs with a second single nucleotide polymorph.

In certain embodiments, the first single nucleotide polymorph and the second single nucleotide polymorph are both present on a continuous single stranded nucleic acid target.

In certain embodiments, the continuous single stranded nucleic acid is conjugated to a label or probe.

In certain embodiments, this disclosure relates to methods of detecting the presence of two single nucleotide polymorph mutations comprising, contacting a particle or surface disclosed herein with a sample comprising a target nucleic acid, wherein the target nucleic acid comprises a first single nucleotide polymorph segment, a second single nucleotide polymorph segment and a spacer segment; providing a target nucleic acid bound to the particle or surface as the first single nucleotide polymorph binding segment of the first single nucleic polymorph binding nucleic acid is hybridized to the first single nucleotide polymorph segment of the target nucleic acid; wherein the first single nucleotide polymorph binding segment has a nucleobase that base pairs with a first single nucleotide polymorph; as the second single nucleotide polymorph binding segment of the second single nucleic polymorph binding nucleic acid is hybridized to the second single nucleotide polymorph segment of the target nucleic acid; wherein the second single nucleotide polymorph binding segment has a nucleobase that base pairs with a second single nucleotide polymorph; and detecting that the target nucleic acid is bound to the particle or surface providing the presence of a first single nucleotide polymorph mutation and a second single nucleotide polymorph mutation in the target nucleic acid in the sample.

In certain embodiments, detecting that the target nucleic acid is bound to the particle is by purifying the particle by flow cytometer and measuring the concentration of labelled particles conjugated to the target nucleic acid. In certain embodiments, measuring the concentration of labelled particles conjugated to the target nucleic acid is by calculating median fluorescence intensity form flow cytometry histograms.

In certain embodiments, the first single nucleotide polymorph mutation and the second single nucleotide polymorph mutation are more than 10, 20, 30, or 40 nucleotide positions from each other. In certain embodiments, the first single nucleotide polymorph mutation and the second single nucleotide polymorph mutation are more than 10 or 20 nucleotide positions from each other and less than 30 or 40 nucleotide positions from each other. In certain embodiments, the target nucleic acid is greater than 40, 50, or 100 nucleotides in length.

In certain embodiments, the first single nucleic polymorph binding nucleic acid is hybridized to 7, 8, to 9 continuous nucleotides in the first single nucleotide polymorph segment, and the second single nucleic polymorph binding nucleic acid is hybridized to one or more continuous nucleotide in the second single nucleotide polymorph segment when compared to the number of nucleotides in the first single nucleic polymorph binding nucleic acid hybridized to the first single nucleotide polymorph segment.

In certain embodiments, the particle or surface binding segment attached to the particle or surface of the first single nucleic polymorph binding nucleic acid is through the 5′ end and the particle or surface binding segment attached to the particle or surface of the second single nucleic polymorph binding nucleic acid is through the 5′ end.

In certain embodiments, the first single nucleic polymorph binding nucleic acid is hybridized to only eight continuous nucleotides of the first single nucleotide polymorph segment, and the second single nucleic polymorph binding nucleic acid is hybridized to only nine continuous nucleotides of the second single nucleotide polymorph segment.

In certain embodiments, the target nucleic acid is messenger RNA or the target nucleic acid is single or double stranded viral RNA or DNA.

In certain embodiments, the target nucleic acid comprises SEQ ID NO: 6 (target with KRAS with L19F and G12C mutations).

In certain embodiments, the target nucleic acid comprises SEQ ID NO: 15 (target of omicron strain with Q498R, N501Y, and Y505H).

### BRIEF DESCRIPTION OF THE SEVERAL VIEWS OF THE DRAWINGS

FIG. 1A illustrates heteromultivalent hybridization, i.e., a homoMV DNA-coated structure containing only one unique oligonucleotide sequence, A, and a heteroMV DNA-coated structure containing two unique oligonucleotide sequences, A and B.

FIG. 1B illustrates the difficulty in tuning binding affinity by adding an additional base pair to a homoMV binding interaction and the ability of a heteroMV structure to more precisely tune the binding affinity of hybridization to achieve maximum specificity.

FIG. 1C illustrates the hypothesized effect of distance between two SNPs on homoMV and heteroMV hybridization specificity.

FIGS. 2A-2D show and illustrate measuring the specificity and cooperativity of heteromultivalent binding using flow cytometry.

FIG. 2A shows the design of the oligonucleotides included in the screen to maximize discrimination factor and best cooperativity factor. Box indicates the position of the SNP in the target sequence, 5′-TGGTAGTTGGAGCTTGTGGCGTAGG (SEQ ID NO: 1), i.e., a no spacer target nucleic acid with a G to T single nucleotide codon mutation resulting in a G12C amino acid change in the KRAS protein. The top 10T oligo, 5′-TTTTTTTTTTCCAACTACCA (SEQ ID NO: 2), is the tuning oligo and the 11S' oligo, 5′-TTTTTTTTTTCGCCACAAGCT (SEQ ID NO: 3), is a single Nucleotide Polymorph (SNP) binding oligo. The 4T oligo, 5′-TTTTTTTTTTCCAA (SEQ ID NO: 5), oligo is the tuning oligo and the 7S oligo, 5′-TTTTTTTTTTACAAGCT (SEQ ID N: 4) is the SNP binding oligo.

FIG. 2B shows a scheme describing a flow cytometry-based assay used to quantify target binding to 5 μm DNA-coated silica particles.

FIG. 2C shows data on measured discrimination factors for 9S, 5T-9S, 6T-9S, and 10S beads.

FIG. 2D shows data on measured median fluorescence intensity values for 8T, 8S, and 8T-8S beads binding the G12C target.

FIGS. 3A-E shows date used for determining the impact of spacer length on heteromultivalent hybridization specificity and cooperativity.

FIG. 3A shows a scheme describing the design of the no spacer target, the internal and terminal short spacer targets, and the internal and terminal long spacer targets including the chemical structures of the PEG spacer molecules.

FIG. 3B shows measured median fluorescence intensity values for 8T-8S beads binding the G12C (B) with no spacer, internal short spacer, internal long spacer, terminal short spacer, and terminal long spacer targets.

FIG. 3C shows data for WT.

FIG. 3D shows data for calculated cooperativity factors for the 8T-8S beads binding the G12C no spacer, internal short spacer, or the internal long spacer targets.

FIG. 3E shows data for calculated discrimination factors.

FIGS. 4A-C illustrate and show data for determining the impact of binding orientation on heteromultivalent hybridization specificity and cooperativity.

FIG. 4A illustrates a scheme describing n=2 beads with head-to-tail, head-to-head, or tail-to-tail orientation binding to targets with or without a spacer region.

FIG. 4B shows data from histograms and measured median fluorescence intensity values for 8T-8S beads with each orientation binding the G12C no spacer, short spacer, and long spacer targets.

FIG. 4C shows data from representative histograms for 8T, 8S, and 8T-8S beads with each orientation binding the G12C no spacer target providing measured cooperativity factors for 8T-8S beads with each orientation binding the G12C no spacer, short spacer, and long spacer targets.

FIGS. 5A-5F show evaluating and detecting the cis/trans relationship of two mutations using heteromultivalent hybridization.

FIG. 5A shows a scheme illustrating the use of head-to-head orientation heteromultivalent DNA-coated beads to distinguish the heterozygous cis mutations mixture from the heterozygous trans mutations mixture. The double mutant target binds the beads multivalently with high affinity, the single mutant target binds monovalently with low affinity, and the no mutant target shows negligible binding.

FIG. 5B shows a scheme of sequences for binding oligos for hybridizing with and identifying the two SNPs KRAS G12C (G to T) and L19F (G to C) in the two binding orientations of 5′-AGCTTGTGCGTAGGCAAGAGTGGCCTTCACG (SEQ ID NO: 6). The oligos 5′-TTTTTTTTTTCCACAAGCT (SEQ ID NO: 7) and 5′-TTTTTTTTTTCACAAGCT (SEQ ID NO: 8) hybridize with the target having the first SNP (S1) with a 5′ poly T segments for attaching to the surface. The oligos 5′-CCACAAGCTTTTTTTTTTT (SEQ ID NO: 9) and 5′-CACAAGCTTTTTTTTTTT (SEQ ID NO: 10) hybridize with the target having the first SNP (S1) with a 3′-poly T segments for attaching to the surface. The oligos 5′-TTTTTTTTTTCGTGAAGGC (SEQ ID NO: 11) and 5′-TTTTTTTTTTCGTGAAGG (SEQ ID NO: 12) hybridize with the target having the second SNP (S2) with a 5′-poly T segments for attaching to the surface.

FIG. 5C shows data on measured median fluorescence intensity values for each bead with head-to-tail orientation binding each of the targets or target combinations in the legend.

FIG. 5D shows data on measured median fluorescence intensity values for each bead with head-to-head orientation binding each of the targets or target combinations in the legend.

FIG. 5E shows measured cis/trans discrimination factors for each bead with head-to-tail orientation.

FIG. 5F shows measured cis/trans discrimination factors for each bead with head-to-head orientation.

FIGS. 6A-6D illustrate and show data on distinguishing different strains of SARS-CoV-2 using heteromultivalent hybridization.

FIG. 6A shows sequences of targets based on the original strains 5′-TCCCAACCCACTAATGGTGTTGGTTACCA (SEQ ID NO: 13), alpha strain with mutation N501Y, 5′-TCCCAACCCACTTATGGTGTTGGTTACCA (SEQ ID NO: 14), and the omicron strain with Q498R, N501Y, and Y505H, 5′-TCCCGACCCACTTATGGTGTTGGTCACCA (SEQ ID NO: 15) strains of SARS-CoV-2 spike protein, with the mutations in each target indicated with arrows.

FIG. 6B shows a scheme describing the binding of an n=1 bead functionalized with an oligo that is fully complementary to the Omicron target and the binding of an n=2 bead functionalized with S1 and S2 oligos that are complementary to the regions of the target containing the Q498R and Y505H mutations but not the N501Y mutation.

FIG. 6C shows and measured median fluorescence intensity values in histograms (D) for the n=1 and 8S1-9S2 n=2 beads binding each target.

FIG. 6D shows measured discrimination factors for the n=1 and 8S1-9S2 n=2 beads binding the omicron target vs. the original target or the omicron target vs. the alpha target.

FIG. 7A illustrates a particle (1) comprising: a single nucleic polymorph binding nucleic acid (2), wherein the single nucleic polymorph binding nucleic acid (2) comprises a single nucleotide polymorph binding segment (3) and a particle binding segment (4) attached to the nanoparticle (1); and wherein the nanoparticle (1) further comprises a tuning nucleic acid (5), wherein the tuning nucleic acid (5) comprises a target binding segment (6) and a particle binding segment (7) attached to the nanoparticle (1); wherein the nanoparticle further comprises a target nucleic acid (8), wherein the target nucleic acid (8) comprises a single nucleotide polymorph segment (9) having a single nucleotide polymorph (12), a target segment (10) and a spacer segment (11); wherein the target nucleic acid (8) is bound to the nanoparticle (1) as the single nucleotide polymorph binding segment (3) of the single nucleic polymorph binding nucleic acid (2) is hybridized to the single nucleotide polymorph segment (9) of the target nucleic acid (8); wherein the single nucleotide polymorph binding segment (3) has a nucleobase that base pairs with the single nucleotide polymorph (12); wherein the target nucleic acid (8) is bound to the nanoparticle (1) as the target binding segment (6) of the tuning nucleic acid (5) is hybridized to the target segment (10) of the target nucleic acid (8).

FIG. 7B illustrates a particle (1) comprising: a first single nucleic polymorph binding nucleic acid (2), wherein the first single nucleic polymorph binding nucleic acid (2) comprises a first single nucleotide polymorph binding segment (3) and a particle binding segment (4) attached to the particle (1); wherein the particle (1) further comprises a second single nucleic polymorph binding nucleic acid (13), wherein the second single nucleic polymorph binding nucleic acid (13) comprises a second single nucleotide polymorph binding segment (14) and a particle binding segment (7) attached to the particle (1); wherein the particle further comprises a target nucleic acid (8), wherein the target nucleic acid (8) comprises a first single nucleotide polymorph segment (9), a second single nucleotide polymorph segment (15) and a spacer segment (11); wherein the target nucleic acid (8) is bound to the particle (1) as the first single nucleotide polymorph binding segment (3) of the first single nucleic polymorph binding nucleic acid (2) is hybridized to the first single nucleotide polymorph segment (9) of the target nucleic acid (8); wherein the first single nucleotide polymorph binding segment (3) has a nucleobase that base pairs with a first single nucleotide polymorph (12); wherein the target nucleic acid (8) is bound to the particle (1) as the second single nucleotide polymorph binding segment (14) of the second single nucleic polymorph binding nucleic acid (13) is hybridized to the second single nucleotide polymorph segment (15) of the target nucleic acid (8); wherein the second single nucleotide polymorph binding segment (14) has a nucleobase that base pairs with a second single nucleotide polymorph (16).

FIG. 8 illustrates pattering segments of binding nucleic acids capable of heteromultivalent hybridization to a particle surface.

### DETAILED DESCRIPTION

Before the present disclosure is described in greater detail, it is to be understood that this disclosure is not limited to embodiments described, and as such may, of course, vary. An “embodiment” refers to an example and is not necessarily limited to such example. It is also to be understood that the terminology used herein is for describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. Patent law in that they are inclusive or open-ended and do not exclude additional, unrecited elements or method steps. The term “comprising” in reference to an oligonucleotide having a nucleic acid sequence refers to an oligonucleotide or peptide that may contain additional 5′ (5′ terminal end) or 3′ (3′ terminal end) nucleotides or N- or C-terminal amino acids, i.e., the term is intended to include the oligonucleotide sequence or peptide sequence within a larger nucleic acid or peptide. “Consisting essentially of” or “consists of” or the like, when applied to methods and compositions encompassed by the present disclosure refers to compositions like those disclosed herein that exclude certain prior art elements to provide an inventive feature of a claim, but which may contain additional composition components or method steps, etc., that do not materially affect the basic and novel characteristic(s) of the compositions or methods, compared to those of the corresponding compositions or methods disclosed herein. The term “consisting of” in reference to an oligonucleotide or peptide having a nucleotide or peptide sequence refers an oligonucleotide or peptide having the exact number of nucleotides or amino acids in the sequence and not more or having not more than a range of nucleotide expressly specified in the claim. For example, “5′ sequence consisting of” is limited only to the 5′ end, i.e., the 3′ end may contain additional nucleotides. Similarly, a “3′ sequence consisting of” is limited only to the 3′ end, and the 5′ end may contain additional nucleotides.

As used herein, the term “about” or “approximately” refers to plus or minus 10 or 20 percent of the recited value, so that, for example, “about 0.125” means 0.125 plus/minus 0.025, and “about 1.0” means 1.0 plus/minus 0.2.

The term “sample” is used in its broadest sense, in that it has chemical makeup that is physical for analysis, i.e., analyte. In one sense it can refer to a nasal fluid, saliva, cough droplets, or expelled droplets of saliva into the air, e.g., produced by speaking, or other lung fluid blood. In another sense, it is meant to include a specimen or culture obtained from any source, as well as biological and environmental samples. Biological samples include bodily fluids, urine, feces, nasal drip, seminal fluid, hair, skin (dead or epithelial layer of skin), finger or toenail clipping, and blood products such as plasma, serum, and the like. Environmental samples include environmental material such as surface matter, soil, water, crystals, and industrial samples. Preferably the sample is from a subject and encompass fluids, solids, tissues, and gases.

The terms, “nucleic acid,” or “oligonucleotide,” refer to a polymer of nucleotides, e.g., DNA, RNA, modified forms, or combinations thereof. The term “nucleotide” or its plural as used herein is interchangeable with modified forms as known in the art. In certain instances, the art uses the term “nucleobase” which embraces naturally-occurring nucleotide and non-naturally-occurring nucleotides which include modified nucleotides. Thus, nucleotide or nucleobase means the naturally occurring nucleobases A, G, C, T, and U and non-naturally occurring nucleobases, for example and without limitations, xanthine, diaminopurine, 8-oxo-N⁶-methyladenine, 7-deazaxanthine, 7-deazaguanine, N′,N′-ethano-2,6-diaminopurine, 5-methylcytosine (mC), 5-(C₃-C₆)-alkynyl-cytosine, 5-fluorouracil, 5-bromouracil, pseudoisocytosine, 2-hydroxy-5-methyl-4-tr-iazolopyridin, isoguanine, and inosine. Methods of making oligonucleotides of a predetermined sequence are well-known. Solid-phase synthesis methods are preferred for both ribonucleotides and deoxyribonucleotides (the well-known methods of synthesizing DNA are also useful for synthesizing RNA). Ribonucleotides can also be prepared enzymatically.

The term “hybridization” refers to the pairing of complementary nucleic acids. Hybridization and the strength of hybridization (i.e., the strength of the association between the nucleic acids) is impacted by such factors as the degree of complementary between the nucleic acids, stringency of the conditions involved, and the G:C ratio within the nucleic acids.

The terms “complementary” and “complementarity” refer to oligonucleotides (i.e., a sequence of nucleotides) related by the base-pairing rules. For example, for the sequence “A-G-T,” is complementary to the sequence “T-C-A.” Complementarity may be “partial,” in which only some of the bases of the nucleic acids are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in detection methods which depend upon binding between nucleic acids.

Target sequences are “targets” in the sense that they are sought to be sorted out from other nucleic acids, consensus sequences compared to a change in the consensus sequence. As used herein, a “mutation,” “mutant,” or the like of a peptide sequence refers to the expression of a variant amino acid(s) within a peptide defined by positions compared to base amino acids within the sequence segment. Due to three codon translation of amino acids from nucleic acid, several three nucleotide codons may express the same amino acid variant. Sometimes the variant is due to a single nucleotide change, and sometimes the variant is due to more than one nucleotide change.

The term “probe” refers to an oligonucleotide (i.e., a sequence of nucleotides) that is capable of hybridizing to oligonucleotide of interest. A probe may be single-stranded or double-stranded, e.g., hairpins. Probes are useful in the detection, identification, and isolation of particular sequences. It is contemplated that any probe is be labeled with any “reporter molecule,” that is detectable in a detection system, including, but not limited to enzyme based (e.g., ELISA, as well as enzyme-based histochemical assays), fluorescent, radioactive, and luminescent systems, including fluorescent dyes and quenchers. Also contemplated are the use of molecular beacons (MB) which are typically 20-30 base pair (bp) oligonucleotide probes with a fluorophore conjugated to the 5′ end and a quencher at the 3′ end. (Heyduk T & Heyduk E, 2002 Nat Biotech, 20:171-176). MBs are designed with 4-7 bps at the 5′ end which are complementary to the bps at the 3′ end. This self-complementary configuration induces the oligonucleotides to form a stem-loop (hairpin) structure so that the fluorophore and the quencher are within close proximity (<7 nm) and fluorescence is quenched. Hybridization of the MBs with the target mRNA opens the hairpin structure and physically separates the fluorophore from the quencher, allowing a fluorescence signal to be emitted upon excitation.

The term “target,” when used in reference to the polymerase chain reaction, refers to the region of nucleic acid bounded by the primers used for polymerase chain reaction. Thus, the “target” is sought to be sorted out from other nucleic acid sequences. A “segment” is defined as a region of nucleic acid within the target sequence.

The term “polymerase chain reaction” (“PCR”) refers to the method of reported in Mullis et al. U.S. Pat. Nos. 4,683,195, 4,683,202, and 4,965,188, that describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured, and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.”

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process itself are, themselves, efficient templates for subsequent PCR amplifications.

As used herein, the term “surface” refers to the outside part of an object. The area is typically of greater than about one hundred square nanometers, one square micrometer, or more than one square millimeter. Examples of contemplated surfaces are on a particle, bead, wafer, array, well, microscope slide, polymer (plastic), metal, or transparent or opaque glass or other material.

The term “conjugated” refers to linking molecular entities through covalent bonds, or by other specific binding interactions, such as due to hydrogen bonding or other van der Walls forces. The force to break a covalent bond is high, e.g., about 1500 pN for a carbon-to-carbon bond. The force to break a combination of strong protein interactions is typically a magnitude less, e.g., biotin to streptavidin is about 150 pN. Thus, a skilled artisan would understand that conjugation must be strong enough to restrict the breaking of bonds in order to implement the intended results.

A “linking group” refers to any variety of molecular arrangements that can be used to bridge or conjugate molecular moieties together. An example formula may be —Rₙ— wherein R is selected individually and independently at each occurrence as: —CRₙRₙ—, —CHRₙ—, —CH—, —C—, —CH₂—, —C(OH)Rₙ, —C(OH)(OH)—, —C(OH)H, —C(Hal)Rₙ—, —C(Hal)(Hal)-, —C(Hal)H—, —C(N₃)Rₙ—, —C(CN)Rₙ—, —C(CN)(CN)—, —C(CN)H—, —C(N₃)(N₃)—, —C(N₃)H—, —O—, —S—, —N—, —NH—, —NRₙ—, —(C═O)—, —(C═NH)—, —(C═S)—, —(C═CH₂)—, which may contain single, double, or triple bonds individually and independently between the R groups. If an R is branched with an Rₙ it may be terminated with a group such as —CH₃, —H, —CH═CH₂, —CCH, —OH, —SH, —NH₂, —N₃, —CN, or -Hal, or two branched Rs may form an aromatic or non-aromatic cyclic structure. It is contemplated that in certain instances, the total Rs or “n” may be less than 100 or 50 or 25 or 10. Examples of linking groups include bridging alkyl groups, alkoxyalkyl, polyethylene glycols, amides, esters, and aromatic groups.

A “label” refers to a detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes. In one example, a peptide “label” refers to incorporation of a heterologous polypeptide in the peptide, wherein the heterologous sequence can be identified by a specific binding agent, antibody, or bind to a metal such as nickel/nitrilotriacetic acid, e.g., a poly-histidine sequence. Specific binding agents and metals can be conjugated to solid surfaces to facilitate purification methods. A label includes the incorporation of a radiolabeled amino acid or the covalent attachment of biotinyl moieties to a polypeptide that can be detected by marked avidin (for example, streptavidin containing a fluorescent marker or enzymatic activity that can be detected by optical or colorimetric methods). Various methods of labeling polypeptides and glycoproteins are known in the art and may be used. Examples of labels for polypeptides include, but are not limited to, the following: radioisotopes or radionucleotides (such as ³⁵S or ¹³¹I) fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as a leucine zipper pair sequences, binding sites for secondary antibodies, metal binding domains, epitope tags), or magnetic agents, such as gadolinium chelates. In some embodiments, labels may be attached by spacer arms of various lengths to reduce potential steric hindrance.

A “fluorescent tag” or “fluorescent dye” refers to a compound that can re-emit electromagnetic radiation upon excitation with electromagnetic radiation (e.g., ultraviolet light) of a different wavelength. Typically, the emitted light has a longer wavelength (e.g., in visible spectrum) than the absorbed radiation. As the emitted light typically occurs almost simultaneously, i.e., in less than one second, when the absorbed radiation is in the invisible ultraviolet region of the spectrum, the emitted light may be in the visible region resulting in a distinctive identifiable color signal. Small molecule fluorescent tags typically contain several combined aromatic groups, or planar or cyclic molecules with multiple interconnected double bonds. Chen et al. report a variety of fluorescent tags that can be viewed across the visible spectrum. Nature Biotechnology, 2019, 37, 1287-1293. The term “fluorescent tag” is intended to include compounds of larger molecular weight such as natural fluorescent proteins, e.g., green fluorescent protein (GFP) and phycobiliproteins (PE, APC), and fluorescence particles such as quantum dots, e.g., preferably having 2-10 nm diameter.

As used herein, “subject” refers to any animal, preferably a human patient, livestock, or domestic pet. In certain embodiments, detection of an analyte further includes calculating the results, recording the data or results from a reproducible computer-readable signal on non-transitory computer readable media and reporting the results to a medical professional.

Unless stated otherwise as apparent from the following discussion, it will be appreciated that terms such as “detecting,” “receiving,” “quantifying,” “mapping,” “generating,” “registering,” “determining,” “obtaining,” “processing,” “computing,” “deriving,” “estimating,” “calculating,” “inferring” or the like may refer to the actions and processes of a computer system, or similar electronic computing device, that manipulates and transforms data represented as physical (e.g., electronic) quantities within the computer system's registers and memories into other data similarly represented as physical quantities within the computer system memories or registers or other such information storage, transmission or display devices. Embodiments of the methods described herein may be implemented using computer software. If written in a programming language conforming to a recognized standard, sequences of instructions designed to implement the methods may be compiled for execution on a variety of hardware platforms and for interface to a variety of operating systems. In addition, embodiments are not described with reference to any programming language. It will be appreciated that a variety of programming languages may be used to implement embodiments of the disclosure.

In some embodiments, the disclosed methods may be implemented using software applications that are stored in a memory and executed by a processor (e.g., CPU) provided on the system. In some embodiments, the disclosed methods may be implanted using software applications that are stored in memories and executed by CPUs distributed across the system. As such, the modules of the system may be a general-purpose computer system that becomes a specific purpose computer system when executing the routine of the disclosure. The modules of the system may also include an operating system and micro instruction code. The various processes and functions described herein may either be part of the micro instruction code or part of the application program or routine (or combination thereof) that is executed via the operating system.

It is to be understood that the embodiments of the disclosure may be implemented in various forms of hardware, software, firmware, special purpose processes, or a combination thereof. In one embodiment, the disclosure may be implemented in software as an application program tangible embodied on a computer readable program storage device. The application program may be uploaded to, and executed by, a machine comprising any suitable architecture. The system and/or method of the disclosure may be implemented in the form of a software application running on a computer system, for example, a mainframe, personal computer (PC), handheld computer, server, etc. The software application may be stored on a recording media locally accessible by the computer system and accessible via a hard wired or wireless connection to a network, for example, a local area network, or the Internet.

It is to be further understood that because some of the constituent system components and method steps depicted in the accompanying figures may be implemented in software, the actual connections between the systems components (or the process steps) may differ depending upon the manner in which the disclosure is programmed. Given the teachings of the disclosure provided herein, one of ordinary skill in the related art will be able to contemplate these and similar implementations or configurations of the disclosure.

### Surfaces and Composition for Detecting Mutations or Multiple Mutations

This disclosure relates to heteromultivalent nucleic acid-functionalized surfaces, such as particles, and uses in optimizing hybridization specificity for targets containing one, two, or more mutations. In certain embodiment, heteromultivalent hybridization enables fine-tuned specificity for a single SNP and dramatic enhancements in specificity for two non-proximal SNPs empowered by cooperative binding. In certain embodiments, use of specified oligo lengths, spacer lengths, and binding orientation are contemplated. In certain embodiments, this disclosure provides for methods of discrimination between heterozygous cis and trans mutations and between different strains of a virus, e.g., the SARS-CoV-2 virus.

In certain embodiments, this disclosure relates to particles or other surfaces comprising: a single nucleic polymorph binding nucleic acid, wherein the single nucleic polymorph binding nucleic acid comprises a single nucleotide polymorph binding segment and a particle or surface binding segment attached to the particle or other surface; wherein the particle or surface further comprises a tuning nucleic acid, wherein the tuning nucleic acid comprises a target binding segment and a particle or surface binding segment attached to the particle or other surface.

In certain embodiments, the surface is a particle, flat surface, well, separated into sections. In certain embodiments, the surface is transparent, translucent, or opaque. In certain embodiments, the surface is glass, plastic, metal or magnetic, e.g., magnetic beads. In certain embodiments, the particle(s) have an average diameter or between 5 to 100 nanometers, or 100 nanometers to 1 micron, or 1 micron to 1 millimeter, or 1 millimeter to 1 centimeter.

In certain embodiments, the particle or surface further comprises a target nucleic acid, wherein the target nucleic acid comprises a single nucleotide polymorph segment having a single nucleotide polymorph, a target segment, and a spacer segment; wherein the target nucleic acid is bound to the particle or surface as the single nucleotide polymorph binding segment of the single nucleic polymorph binding nucleic acid is hybridized to the single nucleotide polymorph segment of the target nucleic acid; wherein the single nucleotide polymorph binding segment has a nucleobase that base pairs with the single nucleotide polymorph; wherein the target nucleic acid is bound to the particle or other surface as the target binding segment of the tuning nucleic acid is hybridized to the target segment of the target nucleic acid.

In certain embodiments, the spacer segment is more than 5, 10, 15 or 20 nucleotides (nt) between the single nucleotide polymorph segment and the target segment of the target nucleic acid. In certain embodiments, the spacer segment is less than 10, 15, 20, 25, 30 or 35 nucleotides (nt) between the single nucleotide polymorph segment and the target segment of the target nucleic acid, i.e., between the closest nucleotides between the two segments in a continuous single strand.

In certain embodiments, the target binding segment that continuously hybridizes with the target is longer than the single nucleotide polymorph binding segment by one nucleotide.

In certain embodiments, this disclosure relates to methods of detecting the presence of a single nucleotide polymorph mutation comprising, contacting particles or surfaces reported herein with a sample comprising a target nucleic acid, wherein the target nucleic acid comprises a single nucleotide polymorph segment having a single nucleotide polymorph, a target segment, and a spacer segment; wherein the target nucleic acid binds the particle or surface as the single nucleotide polymorph binding segment of the single nucleic polymorph binding nucleic acid is hybridized to the single nucleotide polymorph segment of the target nucleic acid; wherein the single nucleotide polymorph binding segment has a nucleobase that base pairs with the single nucleotide polymorph; wherein the target nucleic acid is bound to the particle or surface as the target binding segment of the tuning nucleic acid is hybridized to the target segment of the target nucleic acid; and detecting that the target nucleic acid is bound to the particle or surface providing the presence of a single nucleotide polymorph mutation in the sample.

In certain embodiments, detecting that the target nucleic acid is bound to the particle is by purifying the particle by flow cytometer and measuring the concentration of labelled particles conjugated to the target nucleic acid. In certain embodiments, measuring the concentration of labelled particles conjugated to the target nucleic acid is by calculating median fluorescence intensity form flow cytometry histograms.

In certain embodiments, detecting that the target nucleic acid is bound to the particle or surface providing the presence of a first single nucleotide polymorph mutation and a second single nucleotide polymorph mutation in the target nucleic acid in the sample can be accomplished by one or a combination of the following steps; purifying the particles or surfaces; contacting the particles or surfaces with a probe that hybridizes with a single stranded segment of the target nucleic acid, e.g., in the spacer segment or segments flanking the single nucleotide polymorph binding segment or segment flanking target binding segments of the tuning nucleic acid and detecting the probe; denaturing the target nucleic acid from the particle or surface and amplifying target nucleic acid by PCR, and sequencing the target nucleic acid.

In certain embodiments, the methods further comprise the steps of labeling, isolating, purifying, or amplifying the target nucleic acid, e.g., using PCR.

In certain embodiments, this disclosure contemplates particles or surfaces comprising: a first single nucleic polymorph binding nucleic acid, wherein the first single nucleic polymorph binding nucleic acid comprises a first single nucleotide polymorph binding segment and a particle or surface binding segment attached to the particle or surface; wherein the particle or surface further comprises a second single nucleic polymorph binding nucleic acid, wherein the second single nucleic polymorph binding nucleic acid comprises a second single nucleotide polymorph binding segment and a particle or surface binding segment attached to the particle or surface.

In certain embodiments, the particle or surface further comprises a target nucleic acid, wherein the target nucleic acid comprises a first single nucleotide polymorph segment, a second single nucleotide polymorph segment and a spacer segment; wherein the target nucleic acid is bound to the particle or surface as the first single nucleotide polymorph binding segment of the first single nucleic polymorph binding nucleic acid is hybridized to the first single nucleotide polymorph segment of the target nucleic acid; wherein the first single nucleotide polymorph binding segment has a nucleobase that base pairs with a first single nucleotide polymorph; wherein the target nucleic acid is bound to the particle or surface as the second single nucleotide polymorph binding segment of the second single nucleic polymorph binding nucleic acid is hybridized to the second single nucleotide polymorph segment of the target nucleic acid; wherein the second single nucleotide polymorph binding segment has a nucleobase that base pairs with a second single nucleotide polymorph.

In certain embodiments, the first single nucleotide polymorph and the second single nucleotide polymorph are both present on a continuous single stranded nucleic acid target.

In certain embodiments, the continuous single stranded nucleic acid is conjugated to a label or probe.

In certain embodiments, the continuous single stranded nucleic acid is messenger RNA, or the continuous single stranded nucleic acid is single stranded viral RNA or DNA.

In certain embodiments, the continuous single stranded nucleic acid comprises SEQ ID NO: 6 (target with KRAS with L19F and G12C mutations).

In certain embodiments, the continuous single stranded nucleic acid comprises SEQ ID NO: 15 (target of omicron strain with Q498R, N501Y, and Y505H).

In certain embodiments, this disclosure relates to methods of detecting the presence of two single nucleotide polymorph mutations in a single continuous nucleic acid comprising, contacting a particle or surface disclosed herein with a sample comprising a target nucleic acid, wherein the target nucleic acid comprises a first single nucleotide polymorph segment, a second single nucleotide polymorph segment and a spacer segment; providing a target nucleic acid bound to the particle or surface as the first single nucleotide polymorph binding segment of the first single nucleic polymorph binding nucleic acid is hybridized to the first single nucleotide polymorph segment of the target nucleic acid; wherein the first single nucleotide polymorph binding segment has a nucleobase that base pairs with a first single nucleotide polymorph; as the second single nucleotide polymorph binding segment of the second single nucleic polymorph binding nucleic acid is hybridized to the second single nucleotide polymorph segment of the target nucleic acid; wherein the second single nucleotide polymorph binding segment has a nucleobase that base pairs with a second single nucleotide polymorph; and detecting that the target nucleic acid is bound to the particle or surface providing the presence of a first single nucleotide polymorph mutation and a second single nucleotide polymorph mutation in the target nucleic acid in the sample.

In certain embodiments, detecting that the target nucleic acid is bound to the particle is by purifying the particle by flow cytometer and measuring the concentration of labelled particles conjugated to the target nucleic acid. In certain embodiments, measuring the concentration of labelled particles conjugated to the target nucleic acid is by calculating median fluorescence intensity form flow cytometry histograms.

In certain embodiments, detecting that the target nucleic acid is bound to the particle or surface providing the presence of a first single nucleotide polymorph mutation and a second single nucleotide polymorph mutation in the target nucleic acid in the sample can be accomplished by one or a combination of the following steps; purifying the particles or surfaces; contacting the particles or surfaces with a probe that hybridizes with a single stranded segment of the target nucleic acid, e.g., in the spacer segment or segments flanking the single nucleotide polymorph binding segment or segment flanking target binding segments of the tuning nucleic acid and detecting the probe; denaturing the target nucleic acid from the particle or surface and amplifying target nucleic acid by PCR, and sequencing the target nucleic acid.

In certain embodiments, the spacer segment is more than 5, 10, 15, or 20 nucleotides (nt) between the first single nucleotide polymorph segment and the second single nucleotide polymorph segment of the target nucleic acid. In certain embodiments, the spacer segment is less than 10, 15, 20, 25, 30, or 35 nucleotides (nt) between the first single nucleotide polymorph segment and the second single nucleotide polymorph segment of the target nucleic acid, i.e., between the closest nucleotides between the two segments in a continuous single strand.

In certain embodiments, the first single nucleotide polymorph mutation and the second single nucleotide polymorph mutation are less than 25 or 30 nucleotide positions from each other, and the target nucleic acid is greater than 40, 50, or 100 nucleotides in length.

In certain embodiments, the first single nucleic polymorph binding nucleic acid is hybridized to 7, 8, to 9 continuous nucleotides in the first single nucleotide polymorph segment, and the second single nucleic polymorph binding nucleic acid is hybridized to one more continuous nucleotide in the second single nucleotide polymorph segment when compared to the number of nucleotides in the first single nucleic polymorph binding nucleic acid hybridized to the first single nucleotide polymorph segment.

In certain embodiments, the particle or surface binding segment attached to the particle or surface of the first single nucleic polymorph binding nucleic acid is through the 5′ end and the particle or surface binding segment attached to the particle or surface of the second single nucleic polymorph binding nucleic acid is through the 5′ end.

In certain embodiments, the particle or surface binding segment attached to the particle or surface of the first single nucleic polymorph binding nucleic acid is through the 3′ end and the particle or surface binding segment attached to the particle or surface of the second single nucleic polymorph binding nucleic acid is through the 3′ end.

In certain embodiments, the particle or surface binding segment attached to the particle or surface of the first single nucleic polymorph binding nucleic acid is through the 5′ end and the particle or surface binding segment attached to the particle or surface of the second single nucleic polymorph binding nucleic acid is through the 3′ end.

In certain embodiments, the particle or surface binding segment attached to the particle or surface of the first single nucleic polymorph binding nucleic acid is through the 3′ end and the particle or surface binding segment attached to the particle or surface of the second single nucleic polymorph binding nucleic acid is through the 5′ end.

In certain embodiments, the first single nucleic polymorph binding nucleic acid is hybridized to eight nucleotides the first single nucleotide polymorph segment, and the second single nucleic polymorph binding nucleic acid is hybridized to nine nucleotides the second single nucleotide polymorph segment.

In certain embodiments, the target nucleic acid is conjugated to a label or hybridized probe.

In certain embodiments, the target nucleic acid is messenger RNA or the target nucleic acid is single stranded viral RNA.

In certain embodiments, the target nucleic acid comprises SEQ ID NO: 6 (target with KRAS with L19F and G12C mutations).

In certain embodiments, the target nucleic acid comprises SEQ ID NO: 15 (target of omicron strain with Q498R, N501Y, and Y505H).

In certain embodiments, the target nucleic acid comprises one or more or two or more viral mutations, cancer mutations, cystic fibrosis mutations, Down syndrome, sickle cell anemia, Huntington's disease, Alzheimer's disease, obesity, type II diabetes, hemochromatosis, lactose tolerance, or other disease or conditions associated with mutations.

In certain embodiments, the cancer is breast cancer, and the mutation(s) are in one of the cancer genes BRCA1, BRCA2, CHEK2, PALB2. In certain embodiments, the cancer is colorectal cancer, and the mutation(s) are in one of the cancer genes APC, EPCAM, MLH1, CHEK2, PTEN, STK11, TP53, MUTYH. In certain embodiments, the cancer is endometrial cancer, and the mutation(s) are in one of the cancer genes BRCA1, EPCAM, MLH1, MSH2, MSH6, PMS2, PTEN, STK11. In certain embodiments, the cancer is ovarian cancer, and the mutation(s) are in one of the cancer genes, ATM, BRCA1, BRCA2, BRIP1, EPCAM, MLH1, MSH2, MSH6. In certain embodiments, the cancer is gastric cancer, and the mutation(s) are in one of the cancer genes APC, CDH1, STK11, EPCAM, MLH1, MSH2, MSH6, PMS2. In certain embodiments, the cancer is prostate cancer, and the mutation(s) are in one of the cancer genes ATM, BRCA1, BRCA2, CHEK2, HOXB13, PALB2, EPCAM, MLH1, MSH2, MSH6, PMS2.

In certain embodiments, the disease is Alzheimer's disease (AD), and the mutation(s) are in one of the cancer genes APP, PSEN1, PSEN2.

In certain embodiments, the disease is cystic fibrosis, and the mutation(s) are in cystic fibrosis transmembrane conductance regulator (CFTR) gene, e.g., a F508del mutation and one or more other mutations.

In certain embodiments, this disclosure contemplates that nucleic acids disclosed herein are patterned in tandem on particles or other surfaces for hetero-multivalent hybridization to segments of a target nucleic acid. In certain embodiments, this disclosure relates to methods for controlling the relative position of a series of unique oligonucleotides on a particle surface.

In certain embodiments, this disclosure relates to particles, surfaces and methods of attaching a single nucleic polymorph binding nucleic acid and a tuning nucleic acid to a nanoparticle surface in close proximity comprising: i) providing a nucleic acid complex comprising 1) a single stranded template nucleic acid having template segments and 2) the single nucleic polymorph binding nucleic acid and the tuning nucleic acid, wherein the single nucleic polymorph binding nucleic acid and the tuning nucleic acid hybridize with the template segments, and wherein the single nucleic polymorph binding nucleic acid and the tuning nucleic acid comprise particle or surface binding segments with an anchor or functional group for conjugating the single nucleic polymorph binding nucleic acid and the tuning acid to the surface or the particle or surface; ii) mixing the nucleic acid complex with the particle or surface under conditions such that particle or surface binding segments are conjugated to the particle or surface, e.g., functional groups for attaching to the surface of the nanoparticle react with or interact with the particle or surface, providing a particle or surface coated with the nucleic acid complex; and iii) separating the single stranded template nucleic acid from nucleic acid complex providing a particle or surface coated with a single nucleic polymorph binding nucleic acid and a tuning nucleic acid in close proximity.

In certain embodiments, this disclosure relates to particles, surfaces and methods of attaching a first single nucleic polymorph binding nucleic acid and a second single nucleic polymorph binding nucleic acid to a nanoparticle surface in close proximity comprising: i) providing a nucleic acid complex comprising 1) a single stranded template nucleic acid having template segments and 2) the first single nucleic polymorph binding nucleic acid and the second single nucleic polymorph binding nucleic acid, wherein the first single nucleic polymorph binding nucleic acid and the second single nucleic polymorph binding nucleic acid hybridize with the template segments, and wherein the first single nucleic polymorph binding nucleic acid and the second single nucleic polymorph binding nucleic acid comprise a particle or surface binding segment with an anchor or functional group for conjugating the first single nucleic polymorph binding nucleic acid and the second single nucleic polymorph binding nucleic acid to the surface or the particle or surface; ii) mixing the nucleic acid complex with the particle or surface under conditions such that particle or surface binding segment are conjugated to the particle or surface, e.g., functional groups for attaching to the surface of the nanoparticle react with or interact with the particle or surface, providing a particle or surface coated with the nucleic acid complex; and iii) separating the single stranded template nucleic acid from nucleic acid complex providing a particle or surface coated with a first single nucleic polymorph binding nucleic acid and a second single nucleic polymorph binding nucleic acid in close proximity.

### Heteromultivalency Enables Enhanced Detection of Nucleic Acid Mutations

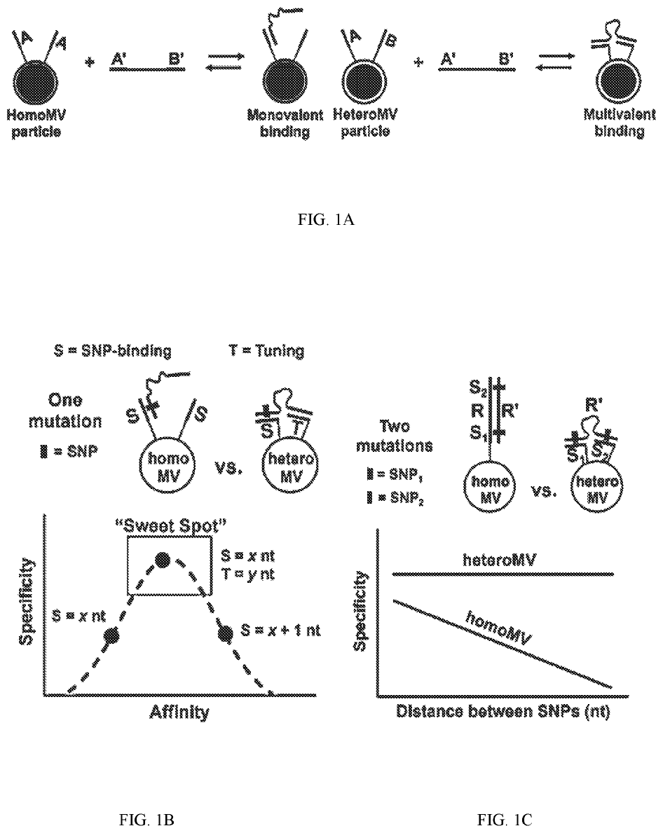

Experiments were performed to determine whether multivalent binding can be used to optimize the specificity of hybridization improving the performance of nucleic acid sensing assays. Target binding can take place on DNA-functionalized surfaces or particles. These homomultivalent DNA-coated structures (homoMV) (FIG. 1A), typically hybridize “monovalently”, forming a single duplex with each target. Heteromultivalent (heteroMV) structures present multiple distinct oligonucleotide sequences can bind multivalently to non-repetitive targets with high avidity. Experiments were performed to determine whether presenting a tuning oligo (T) alongside a SNP-binding oligo (S) can precisely tune target binding affinity and achieve high specificity for a SNP without relying on buffer optimization (FIG. 1B).

Specificity is implicated in applications that require detecting multiple mutations in a single target. For example, haplotype phasing analyses involve distinguishing “cis” and “trans” mutations located on the same or different chromosome copy. Differentiating viral strains also requires optimizing specificity for unique mutations. However, detecting two mutations on a target is difficult to achieve, as monovalent binding probes bind either both sites and the region in between (R′) with low specificity, or bind each mutation separately with no cooperativity. To address this challenge, heteroMV binding was engineered to hybridize cooperatively to two mutations with a non-complementary spacer in between (FIG. 1C). With heteroMV binding, overall affinity for a desired target is enhanced while maintaining low affinity for single mutant or wildtype targets. It is contemplated that specificity significantly increases when two mutations are targeted through heteroMV binding.

HeteroMV DNA-coated silica microparticles presenting two unique oligo sequences (n=2) of different length that bind to single stranded targets containing a complementary region to each oligo were evaluated. The two oligos bind single or double mutant targets in several different orientations while the complementary target regions are directly adjacent or separated by a spacer. A flow cytometry-based assay was used that allows rapid measurement of target binding to each microparticle. HeteroMV binding boosts discrimination for a SNP by a factor of up to 10 over monovalent binding when the length of T is greater than S. Moreover, cooperativity is maximized when the T and S oligos are tuned such that they bind with similar, yet weak affinities. This high cooperativity persists when binding to two sites of a target separated by an up to 15 nucleotides (nt) long spacer region and can be further improved by modifying the binding orientation of the two oligos. Through precise tuning of both specificity and cooperativity the ability to distinguish model heterozygous cis and trans mutations. HeteroMV hybridization was applied to model SARS-CoV-2 targets corresponding to the Original, Alpha, or Omicron strains. Approximately 800-fold binding enhancement was observed for the Omicron target compared to only approximately 12-fold enhancement using homoMV particles. Overall, heteroMV binding greatly expands the potential of DNA hybridization-based assays and DNA nanotechnology by offering highly tunable specificity and cooperativity.

### Measuring the Specificity and Cooperativity of Heteromultivalent Hybridization

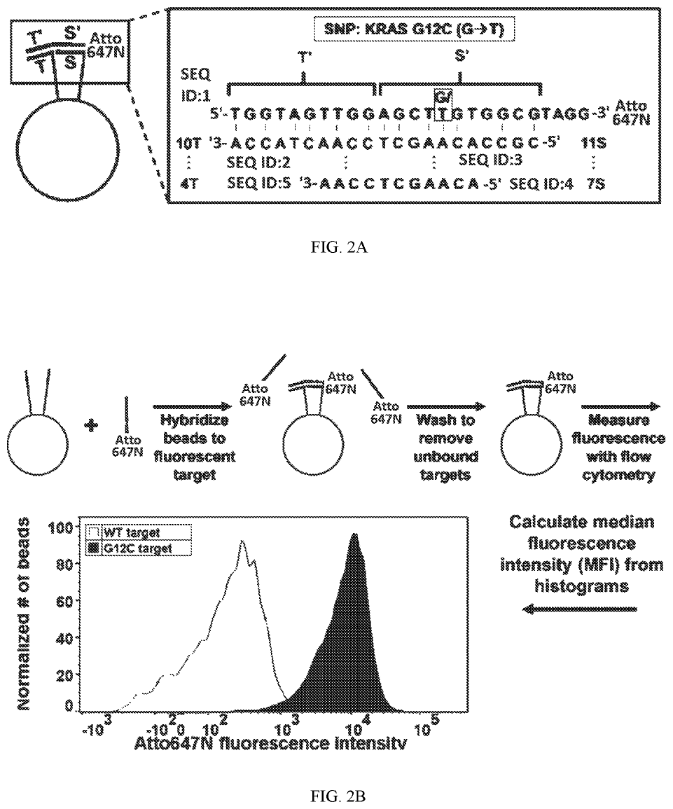

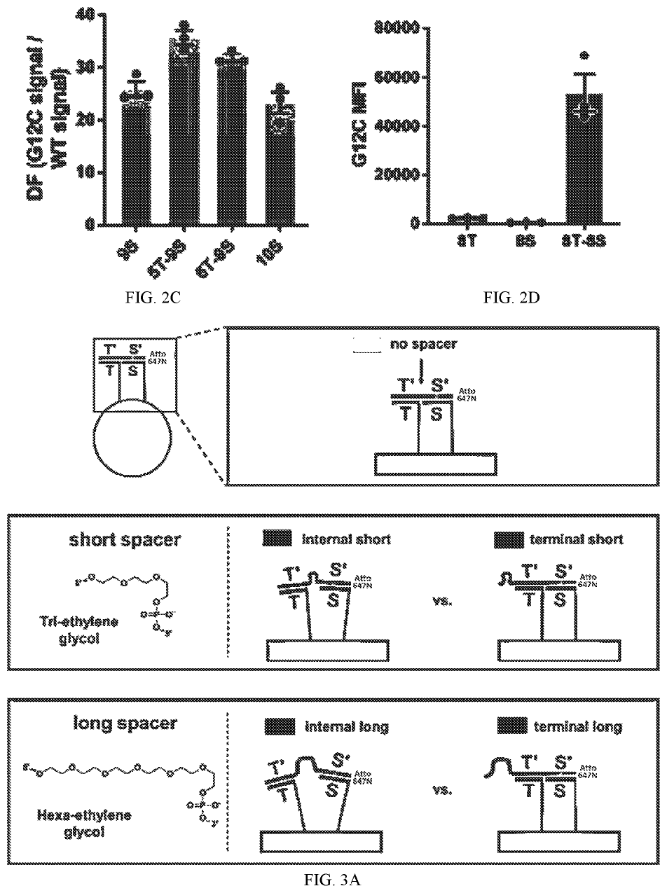

Experiments were performed to evaluated modeling predictions, five S oligos (7-11 nt long, 7S-11S) and seven T oligos (4-10 nt long, 4T-10T) were designed complementary to a 25 nt region of the KRAS genetic sequence that contains the G12C mutation (FIG. 2A). KRAS is an important oncogene and a driver of lung, pancreatic, and colorectal cancers when mutated. The G12C mutant target was perfectly complementary to the S and T oligos, whereas the WT target lacking the mutation binds the S oligo with a single base mismatch and the T oligo with no mismatches. Both targets were modified at their 3′ termini with an Atto647N fluorophore. Each of the S and T oligos contained a T10 polynucleotide linker and a 5′ thiol group to enable conjugation to silica beads. Beads were modified with each possible combination of the S and T oligos, generating a library of 48 unique DNA-coated silica beads. The density of the oligos on the beads were measured by first dissolving the beads in 0.1 M KOH and then using Oligreen™ reagent to quantify the amount of DNA in solution. These measurements revealed that there were about 4.1×10⁴ oligos/m² and an average oligo spacing of about 5 nm, allowing S and T oligos to bind multivalently to the same target. Fluorescence microscopy was also used to image targets hybridized to the beads and confirmed homogeneous binding across the bead surface. A flow cytometry-based assay was designed to measure relative binding of targets to each of the 48 beads. In this assay, the DNA-coated beads were incubated with 1 nM of target in 1×SSC and 0.1% Tween buffer, after which unbound targets were removed through centrifugation and the fluorescence intensity of each individual particle was measured using a flow cytometer (FIG. 2B). Median fluorescence intensities (MFIs) generally increased when the S and/or the T oligo increased in length, confirming that increasing binding affinity results in higher surface occupancy (FIGS. 2C and 2D). To quantify specificity, discrimination factor (DF) values were calculated for each bead mixture by dividing the G12C and WT MFIs. To calculate the equilibrium binding occupancy of the particle-functionalized oligos, one converts the equilibrium binding occupancy to an arbitrary assay signal using inputted maximum and background assay signals, i.e., by comparing the signal when the particles bound the SNP target or the WT target, the discrimination factor (DF) can be calculated.

Consistent with the modeling predictions, the beads presenting the 9S oligo alongside the 5T, 6T, or 7T oligo had the highest DFs. Specifically, the 5T-9S beads yielded about 37% higher specificity compared to the 9S beads, which had the greatest DF of the homoMV beads tested. Importantly, this enhancement was enabled by precise fine-tuning of Keq as the 5T-9S and 6T-9S beads yielded MFIs between that of the 9S and 10S beads (FIG. 2E). In further agreement with the modeling, the screen showed that the 8T-8S beads bound most cooperatively to the G12C target, with almost 40× greater target binding than the average of the 8T and 8S n=1 beads.

### Determining the Impact of Spacer Length on Heteromultivalent Hybridization Specificity and Cooperativity

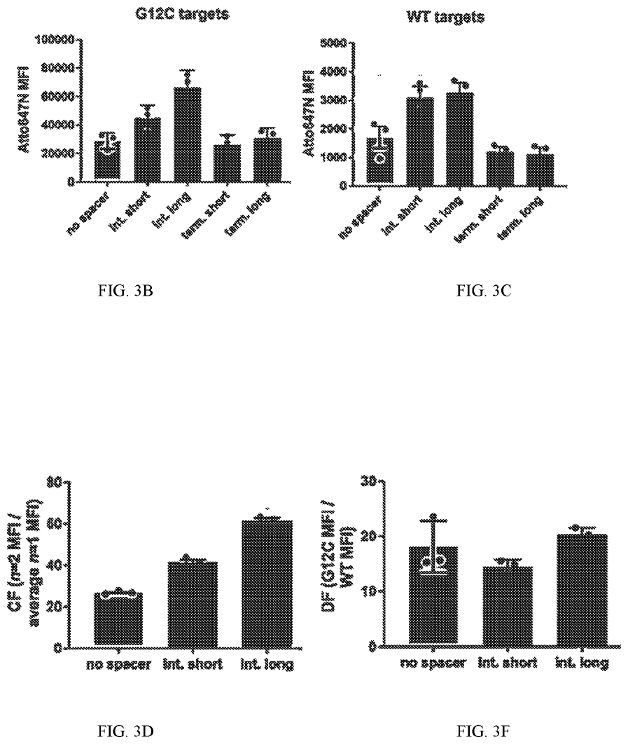

To assess the ability of heteroMV beads to bind with high cooperativity to two non-adjacent regions of a target, several spacer-containing targets were designed and tested. Experiments were designed to test whether hybridization cooperativity and specificity are maintained when the spacer length increases. A tri-ethylene glycol (short) or a hexa-ethylene glycol (long) modification was introduced between the T′ and S′ binding regions (internal) or, as a negative control, at the 5′ terminus of the targets (terminal) (FIG. 3A). Thus, a total of 10 targets were tested with the 8T-8S beads using the flow cytometry-based assay.

The results showed that as internal spacer length increased, more G12C targets bound the beads (FIG. 3B). Inserting a short spacer also enhanced binding to the WT target though the long spacer did not lead to a further increase in binding (FIG. 3C). The terminal spacers did not impact binding to the G12C or WT targets, confirming that the poly-ethylene glycol (PEG) polymer does not chemically influence target binding. The CF of the 8T-8S beads for the G12C targets with different spacer lengths was also calculated by dividing the 8T-8S MFI of beads by the average of the 8T and 8S MFIs of the beads when binding the no spacer target. These calculations revealed significant increases in cooperativity as a function of increasing spacer length (FIG. 3D). The impact of spacer length on specificity was also assessed by calculating the DF of the 8T-8S beads for each target. Interestingly, the internal spacers did not lead to a strong effect on specificity, though there was a significant difference in DF between the short and long spacer targets (FIG. 3E). Surprisingly, 8T and 8S only beads also showed increased binding to the internal spacer-containing targets. This data indicates that heteroMV hybridization allows binding to two spacer-separated regions of a target with increased cooperativity and no loss in specificity compared to a target with no spacer. These results will provide guidance in potential designs of proximity or “AND” logic gate style-assays as well as in diagnostic assays when it is desirable for the tuning oligo to bind a domain (T′) that is not proximal to the SNP site.

### Determining the Impact of Binding Orientation on Heteromultivalent Hybridization Specificity and Cooperativity

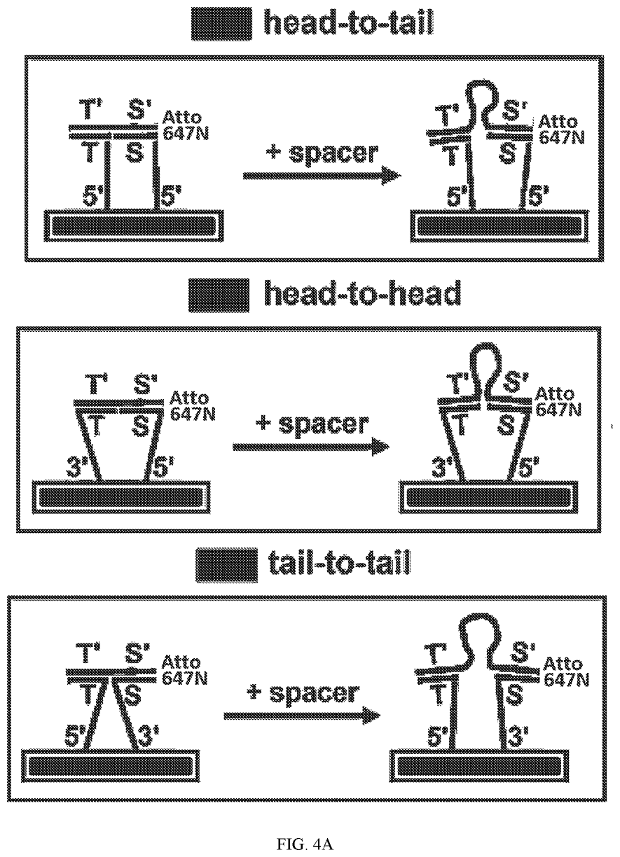

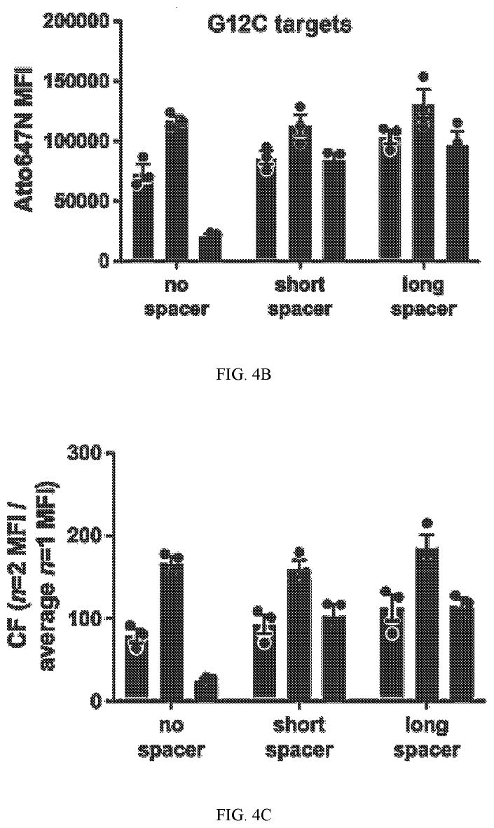

Due to the antiparallel nature of DNA hybridization, the choice of terminus (5′ or 3′) for the anchoring group of the S and T oligos impacts the direction that the oligo binds the target. Therefore, based on the terminus used for each anchor, the two oligos can bind the target in a head-to-tail, head-to-head, or tail-to-tail orientation (FIG. 4A). In this case, head corresponds to the end of the oligo not attached to the particle and tail corresponds to the linker connecting the oligo to the particle. To understand how binding orientation can potentially impact the properties of the binding interaction, 8T-8S beads that bind in the three different orientations were compared using the flow cytometry-based binding assay. Moreover, to investigate how each orientation is influenced by spacer length, the no spacer, short spacer, and long spacer targets were tested with each binding orientation.

When binding the G12C no spacer target, significant differences were observed between the three binding orientations (FIG. 4B). Specifically, the head-to-head binding orientation yielded the highest binding, while the tail-to-tail orientation resulted in a greater than 3-fold reduction in binding compared to the head-to-tail orientation. However, when binding the short or long spacer G12C targets, the tail-to-tail orientation yielded similar binding to the head-to-tail orientation, while the head-to-head orientation still offered slight, non-significant improvements in total binding. Relatedly, the head-to-head orientation beads had a significantly greater than 2-fold increase in CF relative to the head-to-tail orientation beads and a greater than 6-fold increase relative to the tail-to-tail orientation beads when binding the no spacer G12C target (FIG. 4C). The greater average CF for the head-to-head orientation was maintained for the spacer-containing targets, though the enhancement was not significant. The results for the WT target echoed those of the G12C target, and as expected, the anchoring terminus of the oligos did not have a significant effect on n=1 beads binding the G12C no spacer target. Overall, these results validate the importance of binding orientation in tuning binding affinity and cooperativity.

Together, these results can be explained by considering the effects of both the spacing between segments on the bead surface and the base stacking interactions at the interface of the T-T′ and S-S′ duplexes. Based on the distance between the T and S oligos on the surface, different binding orientations can minimize energetic strain during binding depending on linker length and duplex length. For example, if T and S are far apart, then binding the no spacer target in the tail-to-tail orientation might result in significant strain on the T10 linkers. This is consistent with the head-to-head orientation yielding the most avid binding as it binds with only a nick between the two duplexes. In contrast, in the other orientations, the T10 linkers likely interfere with this base-stacking interaction and hence reduce binding affinity and cooperativity.

### Detecting the Cis/Trans Relationship of Two Mutations Using Heteromultivalent Binding.

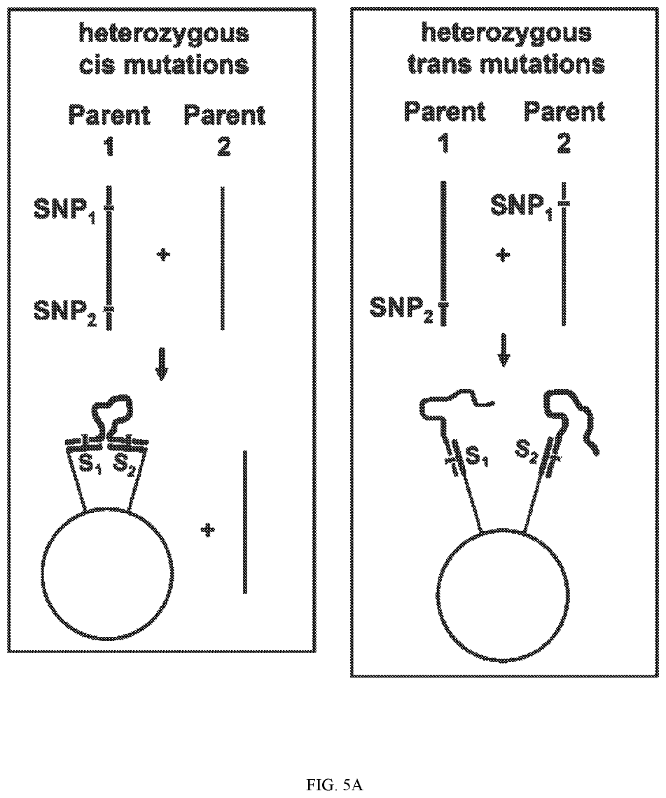

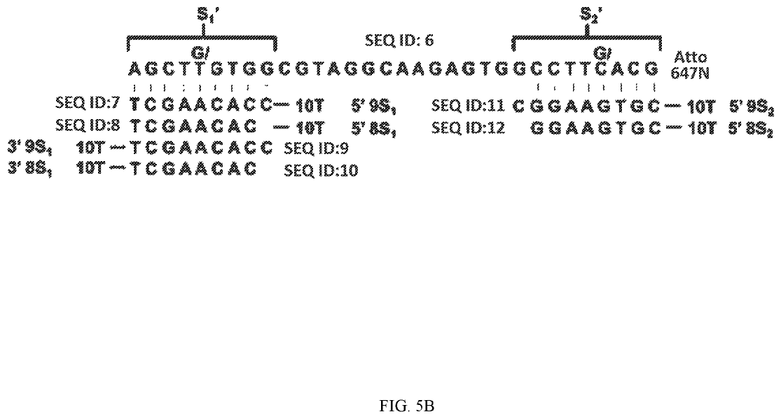

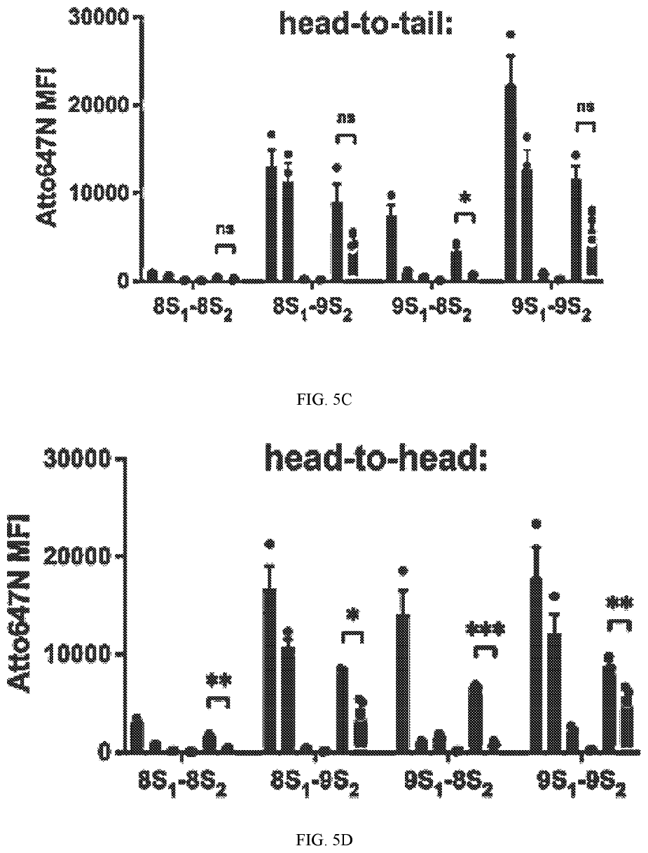

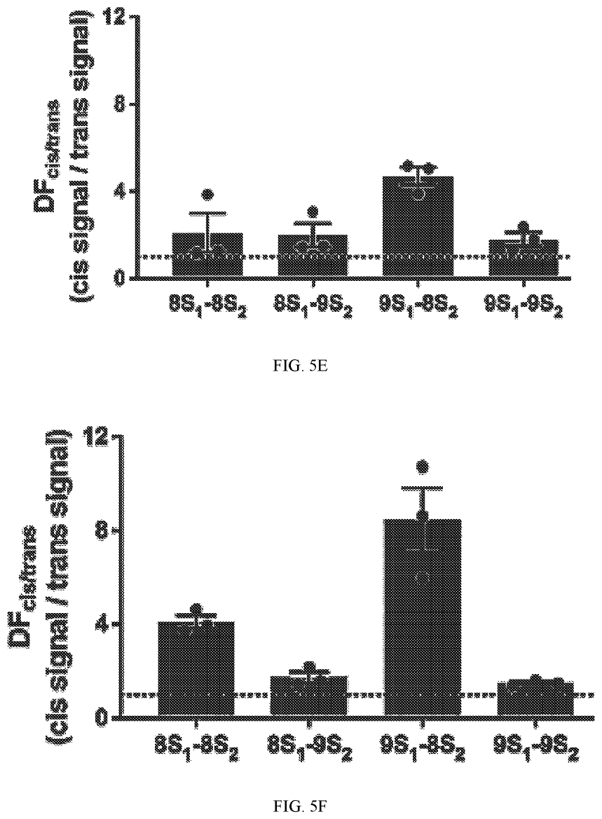

Experiments were conducted to determine whether heteroMV binding can be used to distinguish cis and trans heterozygous mutations (FIG. 5A). This challenging task is significant in medical diagnostics as the presence of two mutations on the same gene copy can alter protein function, while one mutation on each gene copy can yield cells with no functional gene copies. Moreover, cis/trans discrimination is significant. It is contemplated that the presence of two mutations on the same gene copy can alter protein function where one mutation on each gene copy can yield cells with no functional gene copies. Moreover, cis/trans discrimination is significant in genetic counseling in order to track the inheritance of mutations. DNA with 8 and 9 nucleotides S1 and S2 oligos were designed to hybridize in the head-to-tail or head-to-head orientation to a complementary 31 nucleotide target corresponding to a region of the KRAS gene which contains the G12C mutation (SNP1) in the S1′ region and the L19F mutation (SNP2) in the S2′ region (FIG. 5B). Between the S1′ and S2′ regions there are 13-15 non-complementary nucleotides. L19F is a non-canonical mutation that has been found to cause increased tumor proliferation and transforming potential over WT KRAS. This mutation was chosen due to its proximity to the G12C mutation (23 nucleotides away). It is contemplated that binding two mutations that are further apart will still be effective.

Using each combination of the binding oligos, 8 heteroMV beads were synthesized and flow cytometry was used to measure their binding to 1 nM of the four targets, as well as to a 0.5 nM of SNP1/SNP2+0.5 nM of WT1/WT2 target mixture (cis) or a 0.5 nM of SNP1/WT2+0.5 nM of WT1/SNP2 target mixture (trans) (Figured 5C and SD). Bead combinations bound the SNP1/SNP2 target with the greatest affinity and the WT1/WT2 target with the weakest affinity.

The 9S1-8S2 beads with either binding orientation had weak and approximately equal binding to both single mutant targets while showing strong binding to the SNP1/SNP2 target, yielding DF values about 10 for both mutations. Due to this specificity for both mutations and strong binding cooperativity, both the head-to-tail and head-to-head 9S1-8S2 beads bound the cis target combination significantly more than the trans with DF cis/trans values of 4.7 and 8.4, respectively (FIGS. 5E and 5F). Interestingly, both beads containing the 8S2 oligo had higher DF cis/trans values when binding in the head-to-head orientation. Alternatively, beads containing the 9S2 oligo bound the SNP1/SNP2 and WT1/SNP2 targets similarly resulting in poor specificity for SNP1, and had similar DF cis/trans values in both orientations. This suggests that the affinity of the 9S2 oligo for the target is high resulting in low cooperativity binding that is not impacted by a mismatch in the S1′ region. These results offer further evidence that the head-to-head orientation can yield higher binding, particularly when the two immobilized oligos are binding cooperatively. Overall, this screen reveals that heteroMV hybridization enables strong discrimination between cis and trans heterozygous mutations and demonstrates the importance of precisely tuned binding specificity and cooperativity. This result is important as it establishes a hybridization-based approach to distinguish cis/trans mutations without using enzymes or magnetic separation techniques.

### Distinguishing Different Strains of SARS-CoV-2 Using Heteromultivalent Hybridization

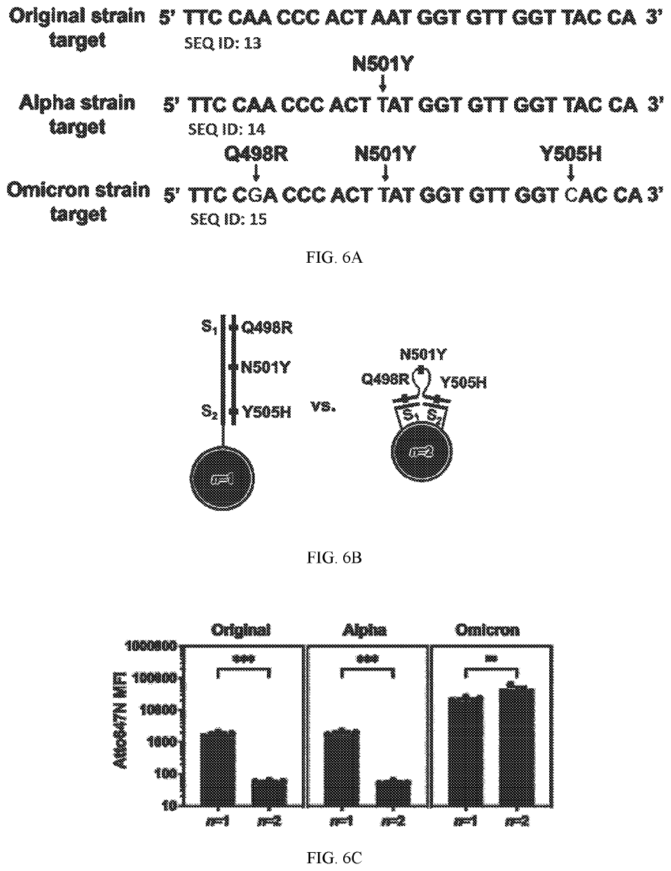

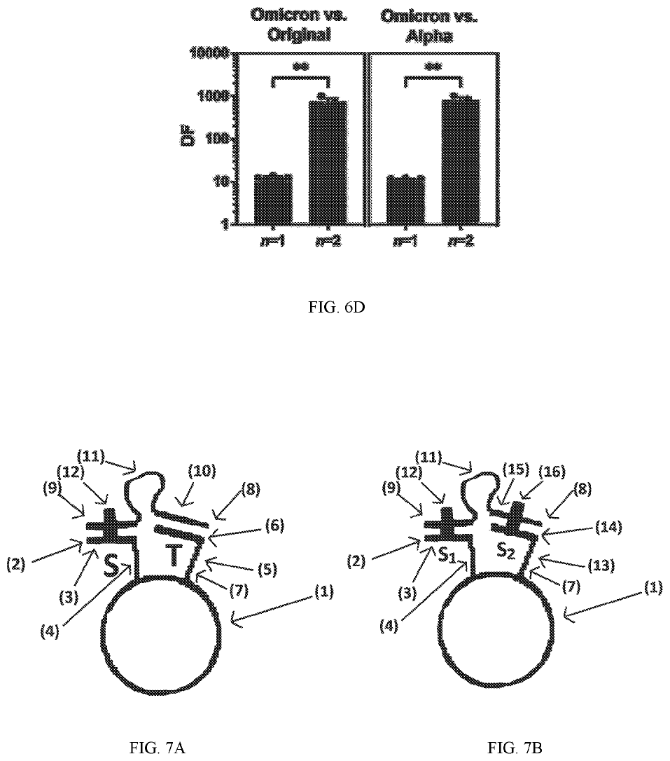

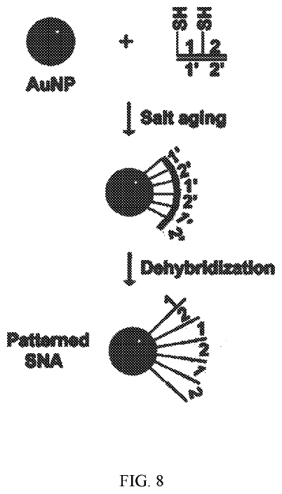

Experiments were designed to determine whether heteroMV hybridization could lead to enhancements in specificity for targets containing two mutations. Model targets corresponding to a 29 nucleotides region of the SARS-CoV-2 spike protein gene were designed to contains three mutations (Q498R, N501Y, and Y505H) in the omicron strain, one mutation in the alpha strain (N501Y), and no mutations in the original strain (FIG. 6A). To hybridize specifically to the omicron strain, 8 and 9 nucleotides S1 and S2 oligos, complementary to the Q498R site and the Y505H site respectively, were designed so that neither overlap with the N501Y mutation shared by the alpha strain (FIG. 6B). Using these oligos, four n=2 beads were synthesized that bound the target in the head-to-head orientation with an 11-13 nucleotide spacer region. As a negative control, n=1 beads functionalized with a 29 nucleotides oligo that is perfectly complementary to the omicron target were also tested (FIG. 6B). Flow cytometry results showed that each of the n=2 beads tested bound to the omicron target with similarly high affinity and showed minimal binding to the alpha and original targets. Meanwhile, compared to the n=2 beads, the n=1 beads yielded an approximately equal MFI when binding the omicron target but bound to significantly more alpha and original targets (FIG. 6C). Importantly, the n=2 beads offered dramatically enhanced specificity for the omicron strain, with the 8S1-9S2 combination bead giving a DFSNP1+SNP2 value of about 800 compared to either of the other targets (FIG. 6D). The n=1 bead had much lower specificity for the Omicron target with DFSNP1+SNP2 values of about 12.

Because the n=1 bead has more total complementarity with the targets, it was surprising that the n=1 and n=2 beads yielded approximately equal omicron target binding. This highlights a general advantage for heteroMV hybridization where each oligo can be shorter in length and therefore less likely to be impacted by these issues. Moreover, the stark differences in specificity between the n=1 and n=2 beads would likely become even greater as the inter-SNP distance increases. In this case, the length of the oligo on the n=1 bead would have to become longer to bind to both SNPs, while the oligos on the n=2 beads would not need to be altered, and instead potentially exhibit stronger and more cooperative binding. Interestingly, the DFSNP1+SNP2 values obtained were even higher than predicted, possibly a result of increased secondary structure for the original and alpha targets relative to the omicron target. This demonstration of rapid and effective identification of the strain of model viral targets using heteroMV hybridization has the potential to significantly impact the fields of diagnostics, medicine, and public health.

1. A particle comprising:
a single nucleic polymorph binding nucleic acid, wherein the single nucleic polymorph binding nucleic acid comprises a single nucleotide polymorph binding segment and a particle binding segment attached to the particle;
wherein the particle further comprises a tuning nucleic acid, wherein the tuning nucleic acid comprises a target binding segment and a particle binding segment attached to the particle.

2. The particle of claim 1, wherein the particle further comprises a target nucleic acid, wherein the target nucleic acid comprises a single nucleotide polymorph segment having a single nucleotide polymorph, a target segment, and a spacer segment;
wherein the target nucleic acid is bound to the particle as the single nucleotide polymorph binding segment of the single nucleic polymorph binding nucleic acid is hybridized to the single nucleotide polymorph segment of the target nucleic acid;
wherein the single nucleotide polymorph binding segment has a nucleobase that base pairs with the single nucleotide polymorph; wherein the target nucleic acid is bound to the particle as the target binding segment of the tuning nucleic acid is hybridized to the target segment of the target nucleic acid.

3. The particle of claim 2 wherein the spacer segment is less than 15 nucleotides (nt) between the single nucleotide polymorph segment and the target segment of the target nucleic acid.

4. The particle of claim 2 wherein the spacer segment is more than 5 nucleotides (nt) between the single nucleotide polymorph segment and the target segment of the target nucleic acid.

5. The particle of claim 1, wherein the target binding segment that continuously hybridizes with the target is longer than the single nucleotide polymorph binding segment by one nucleotide.

6. A method of detecting the presence of a single nucleotide polymorph mutation comprising,
contacting the particle of claim 1 with a sample comprising a target nucleic acid, wherein the target nucleic acid comprises a single nucleotide polymorph segment having a single nucleotide polymorph, a target segment, and a spacer segment;
wherein the target nucleic acid binds the particle as the single nucleotide polymorph binding segment of the single nucleic polymorph binding nucleic acid is hybridized to the single nucleotide polymorph segment of the target nucleic acid;
wherein the single nucleotide polymorph binding segment has a nucleobase that base pairs with the single nucleotide polymorph; wherein the target nucleic acid is bound to the particle as the target binding segment of the tuning nucleic acid is hybridized to the target segment of the target nucleic acid; and
detecting that the target nucleic acid is bound to the particle providing the presence of a single nucleotide polymorph mutation in the sample.

7. A particle comprising: a first single nucleic polymorph binding nucleic acid, wherein the first single nucleic polymorph binding nucleic acid comprises a first single nucleotide polymorph binding segment and a particle binding segment attached to the particle;
wherein the particle further comprises a second single nucleic polymorph binding nucleic acid, wherein the second single nucleic polymorph binding nucleic acid comprises a second single nucleotide polymorph binding segment and a particle binding segment attached to the particle.

8. The particle of claim 7, wherein the particle further comprises a target nucleic acid, wherein the target nucleic acid comprises a first single nucleotide polymorph segment, a second single nucleotide polymorph segment and a spacer segment;
wherein the target nucleic acid is bound to the particle as the first single nucleotide polymorph binding segment of the first single nucleic polymorph binding nucleic acid is hybridized to the first single nucleotide polymorph segment of the target nucleic acid; wherein the first single nucleotide polymorph binding segment has a nucleobase that base pairs with a first single nucleotide polymorph;
wherein the target nucleic acid is bound to the particle as the second single nucleotide polymorph binding segment of the second single nucleic polymorph binding nucleic acid is hybridized to the second single nucleotide polymorph segment of the target nucleic acid; wherein the second single nucleotide polymorph binding segment has a nucleobase that base pairs with a second single nucleotide polymorph.

9. The particle of claim 8, wherein the first single nucleotide polymorph and the second single nucleotide polymorph are both present on a continuous single stranded nucleic acid target.

10. The particle of claim 9, wherein the continuous single stranded nucleic acid is messenger RNA or the continuous single stranded nucleic acid is single stranded viral RNA

11. The particle of claim 9, wherein the continuous single stranded nucleic acid comprises SEQ ID NO: 6 (target with KRAS with L19F and G12C mutations).

12. The particle of claim 11, the continuous single stranded nucleic acid is conjugated to a label.

13. The particle of claim 9, wherein the continuous single stranded nucleic acid comprises SEQ ID NO: 15 (target of omicron strain with Q498R, N501Y, and Y505H).

14. The particle of claim 13, the continuous single stranded nucleic acid is conjugated to a label.

15. A method of detecting the presence of two single nucleotide polymorph mutations comprising,
contacting the particle of claim 7 with a sample comprising a target nucleic acid, wherein the target nucleic acid comprises a first single nucleotide polymorph segment, a second single nucleotide polymorph segment and a spacer segment;
providing a target nucleic acid bound to the particle as the first single nucleotide polymorph binding segment of the first single nucleic polymorph binding nucleic acid is hybridized to the first single nucleotide polymorph segment of the target nucleic acid; wherein the first single nucleotide polymorph binding segment has a nucleobase that base pairs with a first single nucleotide polymorph;
as the second single nucleotide polymorph binding segment of the second single nucleic polymorph binding nucleic acid is hybridized to the second single nucleotide polymorph segment of the target nucleic acid; wherein the second single nucleotide polymorph binding segment has a nucleobase that base pairs with a second single nucleotide polymorph; and
detecting that the target nucleic acid is bound to the particle providing the presence of a first single nucleotide polymorph mutation and a second single nucleotide polymorph mutation in the target nucleic acid in the sample.

16. The method of claim 15, wherein the first single nucleotide polymorph mutation and the second single nucleotide polymorph mutation are more than 10 nucleotide positions from each other.

17. The method of claim 15, wherein
the first single nucleic polymorph binding nucleic acid is hybridized to 7 to 9 continuous nucleotides in the first single nucleotide polymorph segment, and
the second single nucleic polymorph binding nucleic acid is hybridized to one more continuous nucleotide in the second single nucleotide polymorph segment when compared to the number of nucleotides in the first single nucleic polymorph binding nucleic acid hybridized to the first single nucleotide polymorph segment.

18. The method of claim 17, wherein
the particle binding segment attached to the particle of the first single nucleic polymorph binding nucleic acid is through the 5′ end and the particle binding segment attached to the particle of the second single nucleic polymorph binding nucleic acid is through the 5′ end.

19. The method of claim 15, wherein
the first single nucleic polymorph binding nucleic acid is hybridized to eight nucleotides the first single nucleotide polymorph segment, and
the second single nucleic polymorph binding nucleic acid is hybridized to nine nucleotides the second single nucleotide polymorph segment.

20. The method of claim 15, wherein the target nucleic acid is messenger RNA or the target nucleic acid is single stranded viral RNA.

21. The method of claim 15, wherein the target nucleic acid comprises SEQ ID NO: 6 (target with KRAS with L19F and G12C mutations).

22. The method of claim 15, wherein the target nucleic acid comprises SEQ ID NO: 15 (target of omicron strain with Q498R, N501Y, and Y505H).
